(19) United States
(12) Patent Application Publication
(10) Pub. No.: US 2006/0052792 A1
(43) Pub. Date: Mar. 9, 2006

(54) Patella reference device

(75) Inventors: Roland Boettiger, Rietheim-Weilheim (DE); Jean Francois Leitner, Uriage (FR)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/206,655

(22) Filed: Aug. 17, 2005

Related U.S. Application Data
(63) Continuation of application No. PCT/EP04/01806, filed on Feb. 24, 2004.

(30) Foreign Application Priority Data
Feb. 26, 2003 (DE) 103 09 500.4

(51) Int. Cl. A61B 17/58

(52) U.S. Cl. 606/88

(57) ABSTRACT

In order to conduct navigation-assisted knee operations in a simple and optimum manner, a patella reference device is proposed for the determination of the spatial position of a patella of a human knee joint during a navigation-assisted surgical procedure, comprising a base unit that can be secured on the patella, a reference element that can be detected by a detection device and a connecting device for the detachable connection of the base unit and the reference element in a first reference position and in at least one further second reference position differing from the first reference position.

This application is a continuation of international application No. PCT/EP2004/001806 filed on Feb. 24, 2004.

The present disclosure relates to the subject matter disclosed in international application No. PCT/EP2004/001806 of Feb. 24, 2004 and German application No. 103 09 500.4 of Feb. 26, 2003, which are incorporated herein by reference in their entirety and for all purposes.

### BACKGROUND OF THE INVENTION

The invention relates to a patella reference device for the determination of the spatial position of a patella of a human knee joint during a navigation-assisted surgical procedure.

Navigation-assisted surgical procedures are conducted in a variety of ways on the human body. The focal point of such procedures are operations, in which joints of the human body are completely or partially replaced by artificial joints. An example of this is an operation for the replacement of a partially destroyed human knee joint by a knee joint endoprosthesis. In known methods, for preparation of saw cuts on the knee joint a reference element is secured on the femur and a further one to the tibia. By determining the articulation points, in particular also the articulation points of joints adjacent to the knee joint, namely the hip joint and the ankle joint, the saw cuts necessary for insertion of the endoprosthesis can be calculated and performed by navigation-assisted procedure.

Difficulties arise with such operations when the patella is not completely removed, but is retained together with the patellar tendon. In particular, it can occur that the contact surface of the patella, i.e. its rear side, does not optimally fit the artificial knee joint. It must then be worked on accordingly to assure optimum movement of the patella. The guiding principles for working on the rear side of the patella for a surgeon are still primarily his experience and intuition.

Therefore, it is an object of the present invention to propose a patella reference device of the above-described type, with which navigation-assisted knee operations can be simplified and optimised.

### SUMMARY OF THE INVENTION

This object is achieved with a patella reference device of the above-described type, comprising a base unit that can be secured on the patella, a reference element that can be detected by a detection device and a connecting device for the detachable connection of the base unit and the reference element in a first reference position and in at last one further second reference position differing from the first reference position.

The device itself and its configuration have the advantage that after opening an access point to the patella, the patella reference device can be secured on the patella and a movement thereof can be referenced during bending and extension of the knee. In addition, the position of the patella can be determined relative to the reference points on the femur and tibia, from which the leg axis and a relative position to the joint cavity can be determined. In any case, the patella reference device remains on the patella during the entire operation, so that its position remains constantly reproducible. Since the patellar tendon is usually pushed aside in lateral direction to work on the femur and tibia on the opened knee joint, wherein its contact surface then points upwards, it is possible with the present operation to also navigate the patella in its pushed aside position, since the reference element can be connected to the base unit in at least one further reference position. If, for example, the reference element is firstly positioned in a region of the front side of the patella, then it can be detached from the base unit, for example, before the patellar tendon is pushed aside in lateral direction and connected again thereto in a second reference position. This could lead to an arrangement, for example, in which the reference element is then located essentially in a region in front of the rear side of the patella, i.e. in the region of its contact surface. In any case it is possible, and irrespective of a position of the patella, to constantly navigate this in a defined manner before, during or after the surgical procedure. This means that a movement of the patella can be tracked and corresponding corrections can be performed on this, and this can occur until a movement of the patella in conjunction with the artificial knee joint follows a desired path.

The structure of the reference device is particularly simple if the base unit has a first frame element and at least one second frame element, wherein in a release position the at least one second frame element is movable relative to the first frame element, and wherein in a securing position the first frame element and the at least one second frame element are immovably secured on one another. The base unit can be secured on the patella particularly simply with this arrangement.

It is favourable if the first frame element and/or the at least one second frame element comprise a guide means for the respective other frame element for guidance of a relative movement of the first and the at least one second frame element in the release position. An unintentional detachment of the at least two frame elements from one another can thus be prevented. Moreover, a single-handed operation of the reference device is possible with the provided guide means.

It is advantageous if the first frame element is displaceable relative to the at least one second frame element. This enables a particularly small structural height of the reference device to be achieved overall. Moreover, the reference device is particularly simple to operate as a result of this.

It would be conceivable for the guide means to define a rectilinear path. However, it is particularly favourable if the guide means defines a curved path for a relative movement of the first and the at least one second frame element in the release position. This is of particular advantage and allows a particularly small structural height of the reference device when the at least two frame elements are curved. Curved frame elements adjust particularly well to an arched shape of the patella and allow a particularly compact structure of the reference device.

To achieve a simple movement of the reference device from the release position into the securing position, a locking device can be provided for the detachable connection of the first frame element to the at least one second frame element in the securing position. As a result, the at least two frame elements are secured relatively to one another in their position, for example, by a locking projection engaging into a locking recess. With a plurality of locking recesses a plurality of securing positions can then be assumed so that the reference device can be secured on patellae of different size.

The reference device can be operated particularly simply if the locking device comprises an operating element for moving the locking device from the release position into the securing position and vice versa. Thus, a connection between the at least two frame elements can be released and secured again solely by operating the operating element.

To prevent the reference device from being unintentionally detached from the patella, it is advantageous if the locking device assumes the securing position in a normal position.

According to a preferred embodiment of the invention it can be provided that the locking device comprises a biasing element for automatically moving the operating element into the normal position. When the reference device is secured on the patella, then the biasing element, e.g. a spring or any other elastic element, ensures that the reference device cannot be detached from the patella.

In order to assure a defined fixture of the reference device on the patella it is favourable if at least three application points are provided on the base unit for placement on the patella, wherein the at least three application points define a base plane. A plane is defined particularly simply and in a defined manner when three application points are provided. In addition, this also results in an optimum fixture of the reference device on the patella.

A particularly good hold of the reference device on the patella results if at least one of the application points is defined by a point of a spike. This can penetrate the patella a small distance and is securely held there.

The reference device is secured on the patella particularly securely if all the application points are defined by a point of a spike.

To obtain an optimum hold of the reference device on the patella, the first frame element and the at least second frame element respectively bear at least one spike. This enables the patella to be held between the spikes of the at least two frame elements. It is favourable in this case if the spikes are arranged on the at least two frame elements such that the frame elements together with the spikes at least partially engage around the patella, this is possible in particular if the base unit has an essentially U-shaped profile in side view.

In order to fix the base unit particularly securely on the patella, at least one spike can be adjustably arranged relative to a frame element. For example, a spike could be arranged to be rotatable relative to one of the frame elements.

The patella is held particularly securely on the reference device if the application points essentially point towards one another. Particularly in the case where the application points are defined by points of spikes, the spike points essentially point towards one another and hold the patella between them.

It is advantageous if the reference element bears at least three transmission elements detectable by the detection device. This enables a position of the reference element to be clearly determined in the space.

In principle, the transmission elements could be active transmission elements. However, it is favourable if the at least three transmission elements are of passive type. Thus, no power supply of the transmission elements is required.

The transmission elements can be detected particularly simply if they are spheres reflecting electromagnetic radiation.

The at least three transmission elements are preferably arranged so that they define a reference element plane. This allows an orbital movement of the patella to be tracked particularly simply by navigation-assisted procedure.

According to a preferred embodiment of the invention it can be provided that in the first reference position the reference element plane runs parallel to the reference element plane in the at least one second reference position. This means that in the first reference position and in the second reference position the reference element can be connected to the base unit so that the reference element plane constantly runs parallel to itself. It can also run parallel to the base plane, for example, in both positions. This allows the patella to be detected equally well in a laterally pushed aside position as in a natural position of the patella on the knee.

Preferably, the reference element has a reference element base unit and the at least three transmission elements are held on the reference element base unit. This configuration allows the reference element base unit to be shaped so that it can be arranged on the base unit in as space-saving a manner as possible.

It is favourable if the at least three transmission elements are detachably held on the reference element base unit. They can be removed as required-and replaced by other transmission elements.

A particularly simple structure of the reference element results if the reference element base unit has the form of a plane, flat ring. Such a ring is simple to produce and permits a particularly small structural height of the reference device overall. In addition an operating area is only minimally covered.

To enable the base unit and the reference element to be connected particularly simply and securely, it can be provided that the connecting device comprises a first coupling element and a second coupling element, wherein the first coupling element is arranged on the reference element and the second coupling element is arranged on the base unit.

In order to assure a defined relative position between the at least two frame elements, it is favourable if the first coupling element and the second coupling element positively intermesh when the reference device assumes the first and/or the at least one second reference position. Once the shape and the position of the patella relative to the reference element have been determined, the position and shape of the patella can be constantly navigated during movement of the reference element relative to the base unit from the first reference position into the second reference position and vice versa.

A particularly simple structure of the reference device results if the first coupling element is arranged on the reference element base unit and the second coupling element is arranged on the first frame element.

According to a preferred embodiment of the invention it can be provided that the first coupling element is movable relative to the second coupling element in a coupling direction for movement of the first and the second coupling element from a decoupling position, in which the first and the second coupling element are separated from one another, into a coupling position, in which the reference device assumes the first and/or the at least one second reference position. This allows coupling positions and desired reference positions to be clearly defined.

The joining of the base unit and the reference element is particularly simple if the coupling direction runs parallel to the base plane and/or parallel to the reference element plane. This allows firstly the base unit to be secured on the patella and then the reference element to be brought close to the base unit in the coupling direction, e.g. from the lateral or medial direction, when the base plane, for example, runs essentially parallel to a patellar plane defined by the patella. It would also be conceivable to move the reference element from the first reference position into the second reference position without detaching the reference element from the base unit, e.g. by swivelling around a swivel axis, which can run parallel to the coupling direction.

It is advantageous if the first coupling element comprises a recess and the second coupling element comprises a projection that can be inserted into the recess. This results in a particularly simple structure of the two coupling elements and therefore of the entire reference device. Moreover, the number of reference positions can be predetermined by the shape of the recess or projection.

In order to prevent an unintentional detachment of the reference element from the base unit, a locking element can be provided for locking the first and the second coupling element in the coupling position.

A particularly simple operation and also a simple structure of the reference device results if the projection is provided with a thread and the locking element is a nut with an internal thread corresponding to the thread.

The following description of a preferred embodiment of the invention serves for more detailed explanation in conjunction with the drawing.

### BRIEF DESCRIPTION OF THE DRAWINGS

FIG. 1 is a schematic view of a navigated knee operation on a patient;

FIG. 2 is a view from the front of an opened knee joint with reference elements on the tibia, femur and patella;

FIG. 3 is a perspective view of a patella reference device;

FIG. 4 is a side view of a base unit of the patella reference device, and

FIG. 5 is a view of the base unit from FIG. 4 in the direction of arrow A.

### DETAILED DESCRIPTION OF THE INVENTION

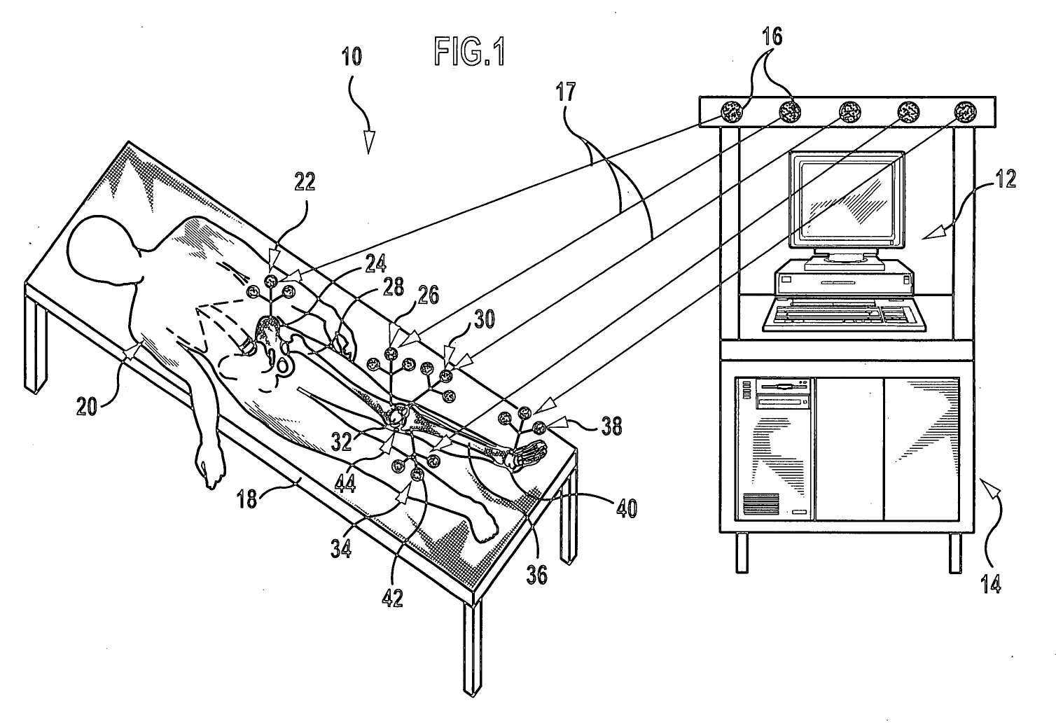

FIG. 1 shows a navigation system given the reference 10 overall for conducting navigated surgical procedures on the human body. It comprises a transmitter and receiver station 14, which is controlled by computer and has several transmitter/receiver units 16 for transmitting and receiving electromagnetic radiation, as well as several reference elements for securing on a patient.

To conduct a navigated knee operation five reference elements are secured on a patient 20 lying on an operating table 18, i.e. a hip marker 22 on the pelvic bone 24, a femur marker 26 in the region of a femur 28 close to the knee, a patella marker 30 according to the invention on the patella 32, a tibia marker 34 in the region of a tibia 36 close to the knee and an ankle joint marker 38 on an ankle joint 40. Each of said markers 22, 26, 30, 34, 38 comprises three identical reflector spheres 42 that reflect electromagnetic radiation 17, which is transmitted by the transmitter/receiver units 16 and which can in turn be received by the transmitter/receiver units 16.

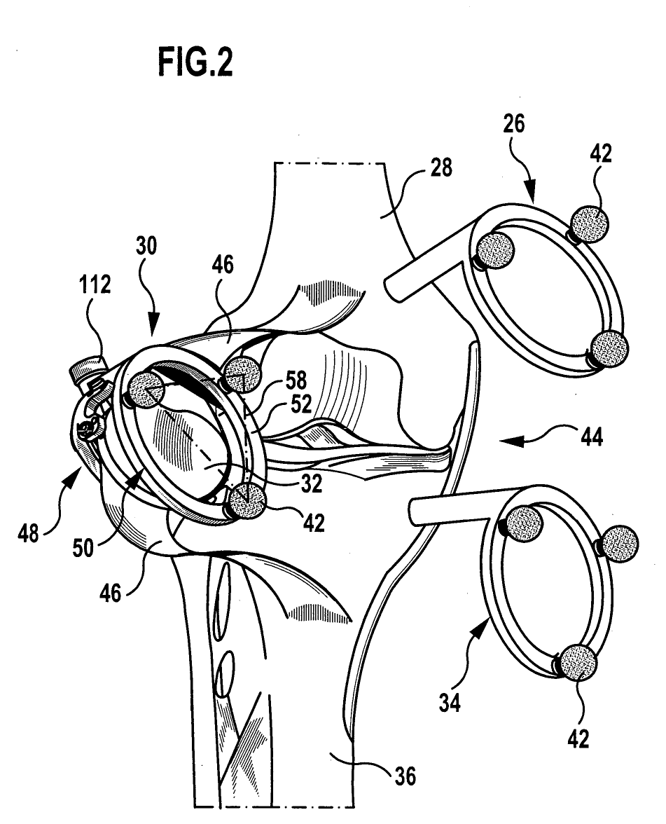

FIG. 2 shows an opened knee joint 44 with the femur marker 26 arranged in the region of the femur 28 close to the knee, with the tibia marker 34 arranged in the region of the tibia 36 close to the knee and with the patella marker 30 arranged on the patella. For work on the knee, as shown in FIG. 2, the patella 32 is pushed aside in lateral direction, so that a patellar tendon 46, which supports the patella 32 and is attached on one side in the region of the femur 28 close to the knee and on the other side in the region of the tibia 36 close to the knee, is twisted slightly.

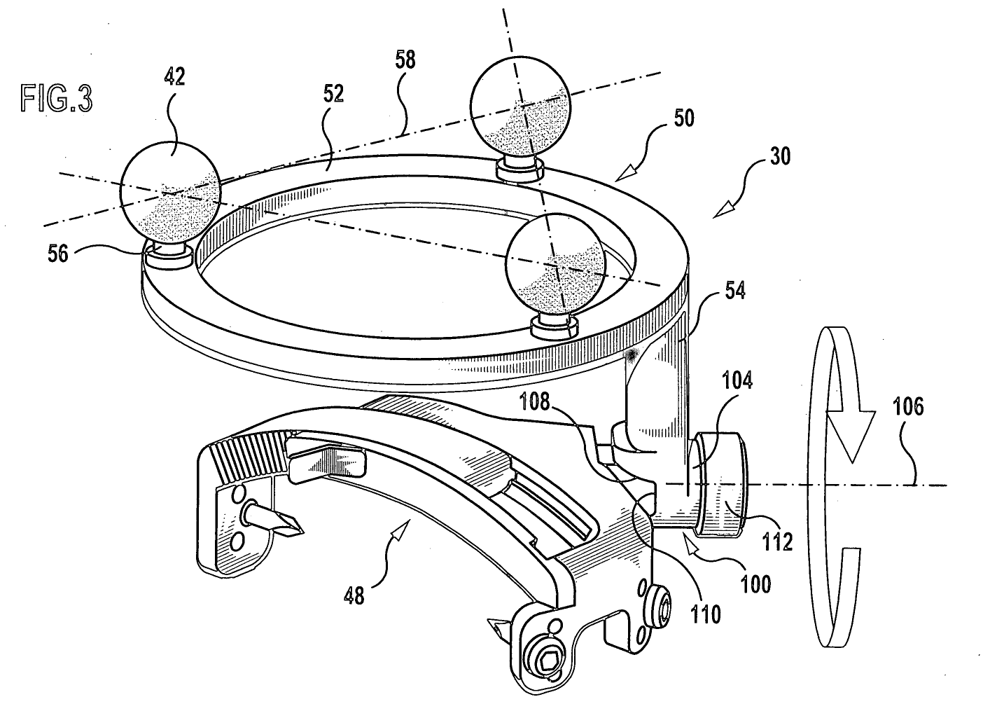

In FIG. 2 the patella marker 30 assumes a second reference position and in FIG. 3 a first reference position, which are defined in more detail below.

A structure of the patella marker 30 shall be explained in more detail below on the basis of FIGS. 3 to 5.

The patella marker 30 comprises a clamp 48 and a reflector attachment 50 as essential basic elements. The reflector attachment 50 is configured essentially in the form of a flat plane ring 52, and a cylindrical spacer bolt 54 extends perpendicularly to a plane defined by the ring away from said attachment. In the opposite direction to the spacer bolt 54, three mountings 56 are evenly distributed on the ring 52 and arranged to project perpendicularly, each having a reflector sphere 42 attached to it. Each reflector sphere 42 is held on a mounting 56 either by a clamp fit or by a releasable snap connection. Centre points of the reflector spheres 42 define a reference element plane 58, which in the present embodiment runs parallel to the plane spanned by the ring 52.

The clamp 48 is configured in essentially two parts. It comprises an essentially U-shaped clamp body 60 with two legs 62 and 64, which are curved in a circular arc shape and run parallel to one another, as well as a plate 66 connecting the two legs 62 and 64. In a plane side face 68 pointing towards the leg 64, the leg 62 has a dovetailed groove 70 curved in a circular arc shape. A corresponding dovetailed projection 72 of a clamping cheek 74 guided between the two legs 62 and 64 is guided in this groove. The clamping cheek 74 can be inserted essentially completely between the two legs 62 and 64 and then completely fills the free space defined by these.

A side face 76 of the leg 64 pointing towards the side face 68 of the leg 62 is essentially smooth. A transversely toothed side face 78 of the clamping cheek 74 lies against it. To fix a relative position between the clamping cheek 74 and the clamp body 60, a release lever 80 is provided, which is disposed to swivel on the leg 64 around a rotational axis 81 and which bears a tooth arrangement (not further shown) corresponding to the toothed side face 78, which can engage in the tooth arrangement of the side face 78 in the position shown in FIG. 5. When the release lever 80 is swivelled in the direction of the arrow 82 shown in FIG. 5, then the positive connection between the tooth arrangement of the release lever and the tooth arrangement of side face 78 is released and the clamping cheek 74 can be displaced relative to the clamp body 60. The clamp 48 then assumes the release position. When the release lever 80 is released, then because of a biasing element (not shown) interacting with the release lever 80 it automatically assumes the securing position again, i.e. the tooth arrangements of the release lever 80 and the side face 78 intermesh again.

To secure the clamp 48 on the patella 32, a holding plate 84 is arranged to project transversely on a free end of the clamping cheek 74. A holding spike 86 provided with a screw thread is held in this holding plate, the point 88 of said spike defining an application point on the patella 32 and said point 88 pointing towards the plate 66. Two holding spikes 92, the longitudinal axes of which are inclined relative to one another and the points 92 of which point approximately towards the point 88 of the holding spike 86, are arranged on the plate 66 in a similar manner. The three points 88 and 92 of the holding spikes 86 and 90 define a base plane 94. When the clamp 48 is joined, i.e. when the clamp body 60 and the clamping cheek 74 are joined, the plate 66 and the holding plate 84 are oriented essentially parallel to one another. The holding spikes 86 and 90 can be adjusted relative to the holding plate 84 or to the plate 66 by rotating the spikes relative to the plates, which is enabled by corresponding threads.

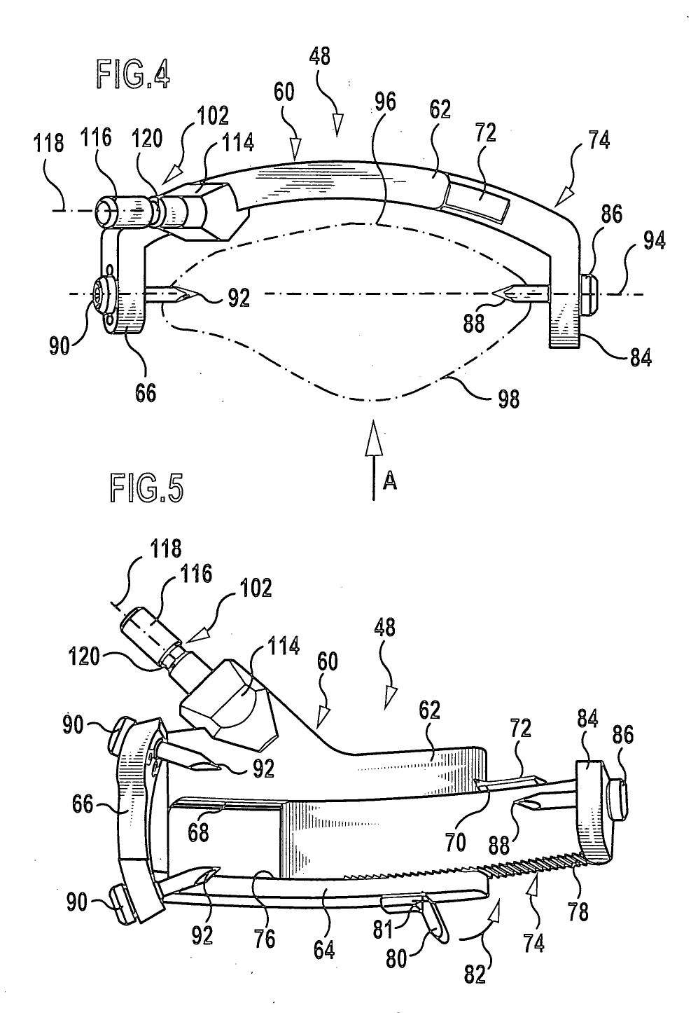

Therefore, as shown in FIG. 4, an essentially U-shaped configuration of the clamp 48 in a side view results, wherein the plate 66 and the holding plate 84 form legs arranged essentially parallel to one another. The curvatures of the legs 62 and 64 as well as of clamping cheek 74 are selected so that the clamp 48 is adapted to fit as tightly as possible onto a curved front side 96 of the patella 32. A rear side 98 of the patella 32, which at the same time forms the contact surface of the patella 32, is freely accessible after securing the clamp 48 to the patella and can be worked on in the desired manner.

To connect the clamp 48 and the reflector attachment 50, these are respectively provided with a coupling element 100 and 102. The coupling element 100 is arranged on a free end of the spacer bolt 54 and comprises a sheath 104 provided with a hole transversely to a longitudinal axis of the spacer bolt 54, wherein the hole defines an axis of symmetry 106. One end of the sheath 104 is cylindrical, while the other end of the sheath bears a groove 108, which widens in a V shape and comprises a groove base 110, which is arranged to run perpendicular to the axis of symmetry 106. Over the cylindrical end of the sheath 104 a knob 112 is held resiliently biased toward the groove 108. It can be pulled back slightly away from the spacer bolt 54 so that locking elements (not shown further) are pulled back transversely to the axis of symmetry 106 from this.

The coupling element 102 is arranged to project laterally on the leg 62 and comprises a clamp body 114 corresponding to the groove 108, which can positively penetrate into the groove 108. The clamp body 114 bears a holding pin 116, which has an axis of symmetry 118 running parallel to the base plane 94. An annular groove 120 is arranged on the holding pin 116.

To join together the clamp 48 and the reflector attachment 50 the holding pin 116 is inserted through the hole of the sheath 104 until the locking elements (not shown) on the coupling element 100 penetrate into the annular groove 102. The clamp body 114 then also penetrates positively into the groove 108 so that the axes of symmetry 106 and 118 coincide. Because of the configuration of the coupling element 100, in particular as a result of the provision of the groove 108, the clamp 48 and the reflector attachment 50 can be interconnected in two different positions, namely a first reference position, as shown in FIG. 3, and a second reference position, as shown in FIG. 2. To move the patella marker 30 from the first reference position into the second reference position, the knob 112 is pulled back until the locking elements disengage from the annular groove 120 and a rotation of the reflector attachment 50 around the axes of symmetry 106 or 118 is possible. After a rotation of 180°, the clamp body 114 can penetrate into the groove 110 again and the locking elements fix the reflector attachment 50 on the clamp 48 in the second reference position by penetrating into the annular groove 120.

In the second reference position, as shown in FIG. 2, the ring 52 surrounds the rear side 98 of the patella 32. Work on the rear side 98 is still possible, since the ring 52 essentially allows free access to the rear side 98.

To record and track an original natural movement of the patella 32, the patella-marker is secured on the patella 32 in the first reference position shown in FIG. 3, i.e. by a relative displacement of the clamp body 60 and the clamping cheek 74 until the points 88 and 92 of the holding spikes 86 and 90 penetrate into the patella 32 so far that the clamp 48 is firmly connected to the patella 32. The patella 32 is pushed aside in lateral direction for work on the femur 28 and the tibia 36, as shown in FIG. 2. To be able to also detect the position of the patella 32 in the pushed aside position, the reflector attachment 50 is swivelled 180° relative to the clamp 48, so that it assumes the second reference position shown in FIG. 2. After the patella 32 has been worked on and parts of the knee joint 42 have been replaced, the patella marker 30 is brought into the first reference position again, and the patella 32 is pushed back into its natural position. A movement of the patella 32 relative to the inserted knee joint parts can be recorded during a bending and extending movement of the knee joint 44.

1. Patella reference device for the determination of the spatial position of a patella of a human knee joint during a navigation-assisted surgical procedure, comprising a base unit that can be secured on the patella, a reference element that can be detected by a detection device and a connecting device for the detachable connection of the base unit and the reference element in a first reference position and in at least one further second reference position differing from the first reference position.

2. Reference device according to claim 1, wherein the base unit has a first frame element and at least one second frame element, wherein in a release position the at least one second frame element is movable relative to the first frame element, and wherein in a securing position the first frame element and the at least one second frame element are immovably secured on one another.

3. Reference device according to claim 2, wherein the first frame element and/or the at least one second frame element comprise a guide means for the respective other frame element for guidance of a relative movement of the first and the at least one second frame element in the release position.

4. Reference device according to claim 2, wherein the first frame element is displaceable relative to the at least one second frame element.

5. Reference device according to claim 3, wherein the guide means defines a curved path for a relative movement of the first and the at least one second frame element in the release position.

6. Reference device according to claim 2, wherein a locking device is provided for the detachable connection of the first frame element to the at least one second frame element in the securing position.

7. Reference device according to claim 6, wherein the locking device comprises an operating element for moving the locking device from the release position into the securing position and vice versa.

8. Reference device according to claim 7, wherein the locking device assumes the securing position in a normal position.

9. Reference device according to claim 8, wherein the locking device comprises a biasing element for automatically moving the operating element into the normal position.

10. Reference device according to claim 1, wherein at least three application points are provided on the base unit for placement on the patella, wherein the at least three application points define a base plane.

11. Reference device according to claim 10, wherein at least one of the application points is defined by a point of a spike.

12. Reference device according to claim 11, wherein all the application points are defined by a point of a spike.

13. Reference device according to claim 11, wherein the first frame element and the at least second frame element respectively bear at least one spike.

14. Reference device according to claim 11, wherein at least one spike is adjustably arranged relative to a frame element.

15. Reference device according to claim 10, wherein the application points essentially point towards one another.

16. Reference device according to claim 1, wherein the reference element bears at least three transmission elements detectable by the detection device.

17. Reference device according to claim 16, wherein the at least three transmission elements are passive transmission elements.

18. Reference device according to claim 16, wherein the transmission elements are spheres reflecting electromagnetic radiation.

19. Reference device according to claim 16, wherein the at least three transmission elements define a reference element plane.

20. Reference device according to claim 19, wherein in the first reference position the reference element plane runs parallel to the reference element plane in the at least one second reference position.

21. Reference device according to claim 16, wherein the reference element has a reference element base unit and the at least three transmission elements are held on the reference element base unit.

22. Reference device according to claim 21, wherein the at least three transmission elements are detachably held on the reference element base unit.

23. Reference device according to claim 21, wherein the reference element base unit has the form of a plane, flat ring.

24. Reference device according to claim 1, wherein the connecting device comprises a first coupling element and a second coupling element, wherein the first coupling element is arranged on the reference element and the second coupling element is arranged on the base unit.

25. Reference device according to claim 10, wherein the connecting device comprises a first coupling element and a second coupling element, wherein the first coupling element is arranged on the reference element and the second coupling element is arranged on the base unit.

26. Reference device according to claim 16, wherein the connecting device comprises a first coupling element and a second coupling element, wherein the first coupling element is arranged on the reference element and the second coupling element is arranged on the base unit.

27. Reference device according to claim 24, wherein the first coupling element and the second coupling element positively intermesh when the reference device assumes the first and/or the at least one second reference position.

28. Reference device according to claim 24, wherein the first coupling element is arranged on the reference element base unit and the second coupling element is arranged on the first frame element.

29. Reference device according to claim 24, wherein the first coupling element is movable relative to the second coupling element in a coupling direction for movement of the first and the second coupling element from a decoupling position, in which the first and the second coupling element are separated from one another, into a coupling position, in which the reference device assumes the first and/or the at least one second reference position.

30. Reference device according to claim 29, wherein the coupling direction runs parallel to the base plane and/or parallel to the reference element plane.

31. Reference device according to claim 24, wherein the first coupling element comprises a recess and the second coupling element comprises a projection that can be inserted into the recess.

32. Reference device according to claim 24, wherein a locking element is provided for locking the first and the second coupling element in the coupling position.

33. Reference device according to claim 31, wherein the projection is provided with a thread and the locking element is a nut with an internal thread corresponding to the thread.
